(19) United States
(12) Patent Application Publication
(10) Pub. No.: US 2008/0015576 A1
(43) Pub. Date: Jan. 17, 2008

(54) LARGE DIAMETER BONE ANCHOR ASSEMBLY

(76) Inventor: Dale E. Whipple, East Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/741,151

(22) Filed: Apr. 27, 2007

Related U.S. Application Data
(60) Provisional application No. 60/796,043, filed on Apr. 28, 2006.

(51) Int. Cl. A61B 17/68

(52) U.S. Cl. 606/60

(57) ABSTRACT

Bone anchor assemblies having a large diameter for fixing a spinal connection element to bone and methods of assembly are described. The assembly includes a receiver member for receiving the spinal connection element, a bone-engaging shank for engaging bone and a sleeve member for retaining the head of the shank within the receiver member.

### CONTINUING DATA

This application claims the benefit of U.S. Provisional Patent Application Ser. No. 60/796,043, entitled “Large Diameter Bone Anchor Assembly”, filed Apr. 28, 2006, which is hereby incorporated herein by reference.

### BACKGROUND

Spinal connection systems may be used in orthopedic surgery to align and/or fix a desired relationship between adjacent vertebrae. Such systems typically include a spinal connection element, such as a relatively rigid fixation rod or plate or a dynamic connector, that is coupled to adjacent vertebrae by attaching the element to various anchoring devices, such as hooks, bolts, wires, or screws. The spinal connection element can have a predetermined contour that has been designed according to the properties of the target implantation site, and once installed, the spinal connection element holds the vertebrae in a desired spatial relationship, either until desired healing or spinal fusion has taken place, or for some longer period of time.

Spinal connection elements can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a spinal connection element receiving portion, which, in spinal rod applications, is usually in the form of a U-shaped slot formed in the head for receiving the rod. A set-screw, plug, cap or similar type of closure mechanism, may be used to lock the connection element into the connection element receiving portion of the pedicle screw. In use, the shank portion of each screw may be threaded into a vertebra, and once properly positioned, a connection element may be seated through the spinal connection element receiving portion of each screw and the connection element is locked in place by tightening a cap or similar type of closure mechanism to securely interconnect each screw and the connection element. Other anchoring devices also include hooks and other types of bone screws.

In certain procedures, such as those in the lumbar or sacral spine, it may be necessary to use a larger diameter pedicle screw capable of carrying large loads or engaging large pedicles. A difficulty in using a larger diameter screw comes from the corresponding increase in the size of the receiver head to accommodate the larger diameter screw shank, since the shank is usually assembled from the top through the opening at the proximal end of the receiver head. The increased size of the receiver head can interfere with the bony anatomy and can limit the polyaxial range of motion of the screw head. Another problem associated with manufacturing large diameter top-loading screws is that the opening in the receiver head has to be larger to accept the larger diameter screw shank, which creates the need for a larger closure mechanism. It is desirable to maintain the same size opening in the receiver head such that the same size closure mechanisms can be used. Accordingly, a larger diameter polyaxial screw is needed which is not top-loading.

### SUMMARY

Disclosed herein are embodiments of a bottom-loading bone anchor assembly having a large diameter shank. In one embodiment, a bone anchor assembly for engagement to a connection element includes a bone-engaging shank having a head at a proximal end, a receiver member having an opening at the proximal end for receiving the connection element and an expandable socket, the expandable socket expandable from a first configuration in which the diameter of the expandable socket is less than the size of the head of the shank to a second configuration in which the diameter of the expandable socket is greater than or equal to the size of the head of the shank; and a sleeve member positionable about the expandable socket, the sleeve member having an inner surface sized and shaped to inhibit expansion of the expandable socket from expanding when positioned about the expandable socket and thereby retains the head of the shank within the receiver member.

A method of assembling a bone anchor includes expanding an expandable socket of a receiver member to advance a head of a bone-engaging shank into the receiver member; and positioning a sleeve member around the expandable socket of the receiver member to inhibit expansion of the expandable socket and retain the head of the bone-engaging shank within the receiver member.

### BRIEF DESCRIPTION OF THE FIGURES

These and other features and advantages of the bone anchor assembly and methods disclosed herein will be more fully understood by reference to the following detailed description in conjunction with the attached drawings in which like reference numerals refer to like elements through the different views. The drawings illustrate principles of the bone anchor assembly and methods disclosed herein and, although not to scale, show relative dimensions.

FIG. 1A illustrates an exploded view of a large diameter bone anchor assembly.

FIG. 1B illustrates a side view of the bone anchor assembly shown in FIG. 1A.

FIG. 1C illustrates a cross-section of the bone anchor assembly shown in FIG. 1B.

FIG. 2A illustrates a perspective view of the sleeve member of the bone anchor assembly shown in FIG. 1A.

FIG. 2B illustrates a cross-section view of the sleeve member of the bone anchor assembly shown in FIG. 2A.

FIG. 2C illustrates a top view of the sleeve member shown in FIG. 2A.

FIG. 3A illustrates a perspective view of the receiver member of the bone anchor assembly shown in FIG. 1A.

FIG. 3B illustrates a side view of the receiver member shown in FIG. 3A with hidden lines.

FIG. 3C illustrates a cross-section view of the receiver member shown in FIG. 3A.

FIG. 4A illustrates a perspective view of the compression member of the bone anchor assembly shown in FIG. 1A.

FIG. 4B illustrates a side view of the compression member shown in FIG. 4A.

FIG. 4C illustrates a cross-section view of the compression member shown in FIG. 4A.

### DETAIL DESCRIPTION OF EXEMPLARY EMBODIMENTS

Certain exemplary embodiments will now be described to provide an overall understanding of the principles of the structure, function, manufacture, and use of the large diameter bone anchor assembly and methods disclosed herein. One or more examples of these embodiments are illustrated in the accompanying drawings. Those of ordinary skill in the art will understand that the large diameter bone anchor assembly and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

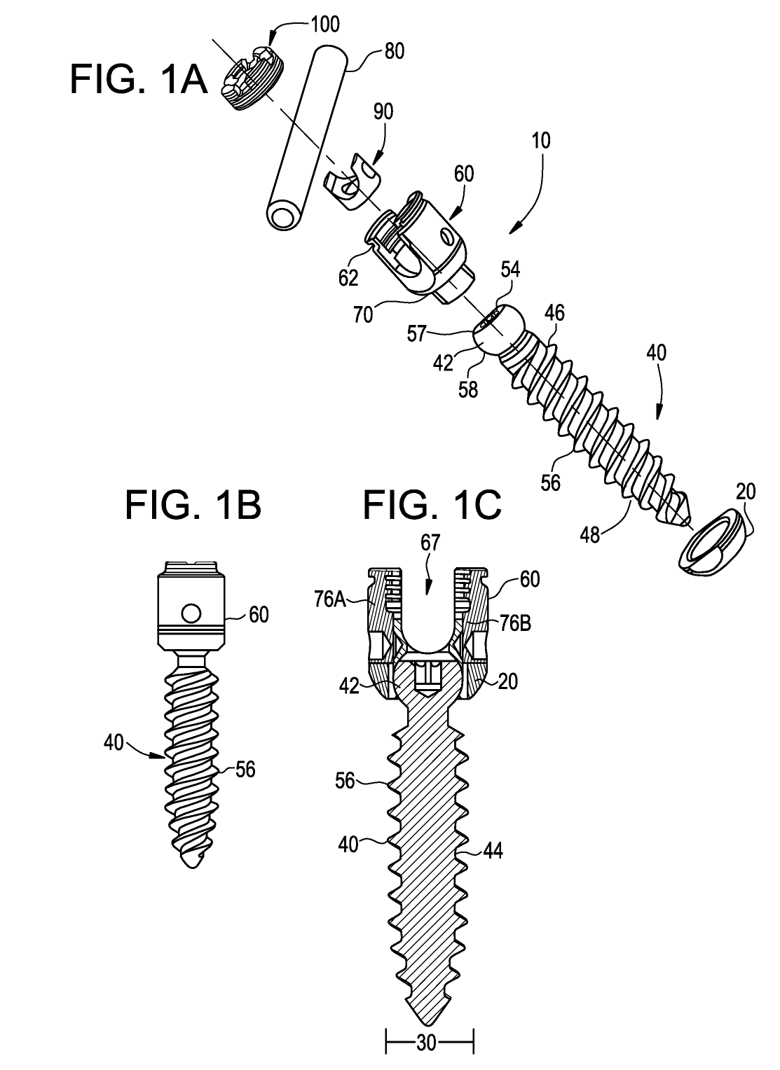

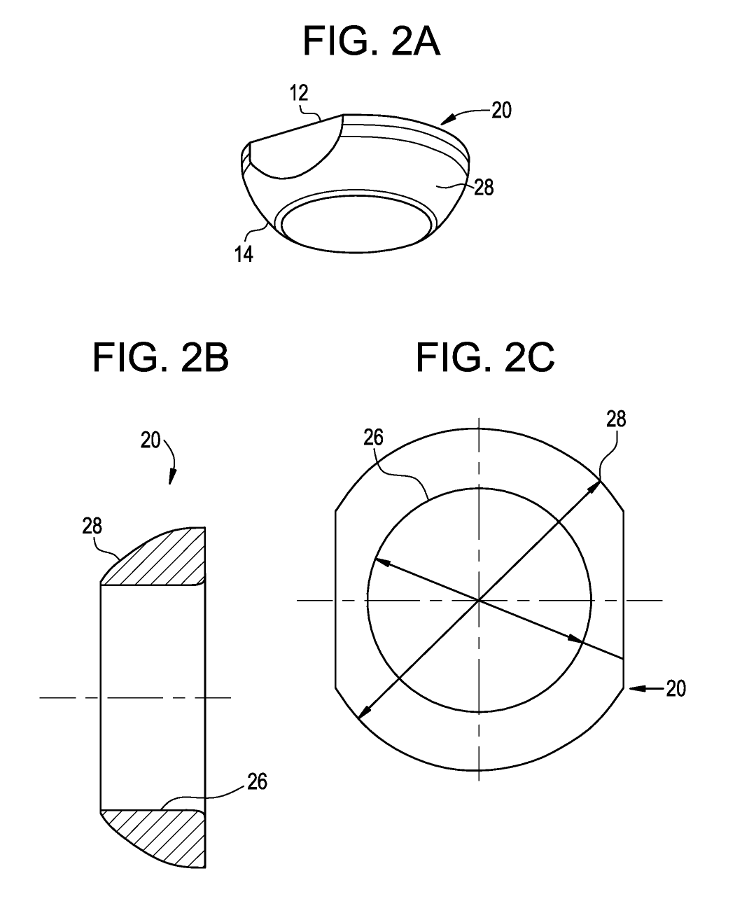

FIGS. 1-4 illustrate an exemplary embodiment of a bottom-loading large diameter bone anchor assembly. The exemplary bone anchor assembly 10 may be employed to engage one or more spinal connection elements to bone. For example, bone anchor assembly 10 may be employed to fix a spinal plate, rod (rigid or dynamic), and/or cable to a vertebra of the spine. Although the exemplary bone anchor assembly 10 described below is designed primarily for use in spinal applications, one skilled in the art will appreciate that the structure, features, and principles of the exemplary bone anchor assembly 10, as well as the other exemplary embodiments described below, may be employed to couple any type of orthopedic implant to any type of bone or tissue. Non-limiting examples of applications of the bone connection anchor assembly 10 described herein include long bone fracture fixation/stabilization, small bone stabilization, lumbar spine as well as thoracic stabilization/fusion, cervical spine compression/fixation, non-fusion applications including facet replacement and dynamic posterior systems, as well as skull fracture/reconstruction plating.

The illustrated exemplary bone anchor assembly 10 includes a bone-engaging shank 40 configured for engaging bone, a receiver member 60 for receiving a spinal connection element, and a sleeve member 20 for retaining the shank 40 within the receiver member 60. The bone-engaging shank 40 extends from a proximal end 46 to a distal end 48 along a longitudinal axis. An outer surface 44 of the bone-engaging shank 40 extends between the proximal end 46 and the distal end 48. The outer surface 44 of the bone-engaging shank 40 may include one or more bone engagement mechanisms to facilitate gripping engagement of the bone anchor assembly 10 to bone. In the illustrated exemplary embodiment, for example, the bone-engaging shank 40 includes an external thread 56. The external thread 56 may extend along at least a portion of the bone-engaging shank 40. For example, in the illustrated exemplary embodiment, the external thread 56 extends from the distal end 48 to the proximal end 46 of the bone-engaging shank 40. One skilled in the art will appreciate that bone engagement mechanisms other than external thread 56 may be employed, including, for example, one or more annular ridges, multiple threads, dual lead threads, variable pitched threads, and/or any other conventional bone engagement mechanism. In the illustrated exemplary embodiment, the shank diameter 30 of bone-engaging shank 40 may be defined by the major diameter of external thread 56.

The proximal end 46 of the exemplary bone-engaging shank 40 has a head 42 configured to fit within the receiver member 60 and to facilitate adjustment of the shank 40 relative to the receiver member 60. For example, the head 42 may be generally spherical in shape to permit pivoting of the bone-engaging shank 40 relative to the receiver member 60. In the illustrated exemplary embodiment, for example, the head 42 may be in the shape of a truncated sphere having a generally planar proximal surface 57 and a generally hemispherically shaped distal surface 58. The head 42 of the shank 40 may have surface texturing, knurling, and/or ridges. A drive feature 54 may be located internally or externally on the head 42 of the shank 40.

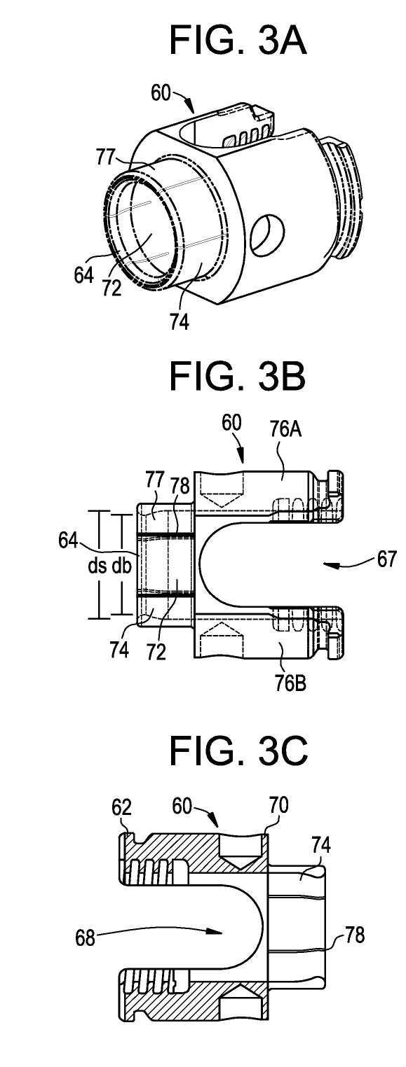

Referring to FIGS. 1A, 3A,B, the receiver member 60 of the exemplary bone anchor assembly 10 includes a proximal end 62 having a cylindrical opening 67 leading to recess 68, and a distal end 70 having an expandable socket 72. The receiver member 60, in certain exemplary embodiments, may be configured to receive a spinal connection element and couple the spinal connection element to the bone anchor assembly. In the exemplary embodiment, for example, the recess 68 of the receiver member 60 may be sized and shaped to receive a spinal rod 80, as illustrated in FIG. 1A. For example, the receiver member 60 has a generally U-shaped cross-section defined by two legs 76A and 76B separated by recess 68. Each leg 76A, 76B is free at the proximal end 62 of the receiver member 60. In the exemplary embodiment, for example, the inner surfaces of the legs 76A, 76B are threaded to mate with a corresponding thread on the closure mechanism shown as a setscrew. The exemplary spinal rod 80 may be seated within the recess 68 by aligning the spinal rod 80 and the recess 68, and advancing the spinal rod 80 between the legs 76A, 76B into the recess 68. The configuration of recess 68 of the receiver member 60 may be varied to accommodate the type, size and shape of spinal connection element employed.

In the exemplary embodiment, the distal end 70 of the receiver member 60 forms an expandable socket 72 having walls 74 forming bore 64 of the receiver member 60. The walls 74 expand to allow at least a portion of a bone anchor assembly, such as the head 42 of the shank 40 to pass through the bore 64 into the expandable socket 72. For example, the head 42 of the shank 40 may be inserted in the proximal direction through the bore 64 of the receiver member 60, as illustrated in FIG. 1A to expand the expandable socket 72. The diameter of the bore 64 is greater than the diameter of the cylindrical opening 67 of the receiver member at the proximal end 62. The expandable socket 72 expands from a first configuration in which the diameter is less than the diameter of the head 42 of the shank 40 to a second configuration in which the diameter is greater than or equal to the diameter of the head 42 of the shank 40. The diameter of the expandable socket 72 in the second configuration is greater than the diameter of both the bore 64 and the opening 67. In one embodiment, the walls 74 have slits 78 which permit the walls 74 to expand around the head 42 of the shank 40. In some exemplary embodiments, the inner surface 79 of the walls 74 may be generally spherical in shape and may have a curvature analogous to the distal surface 58 of the head 42 of the shank 40 and permit pivoting of the bone-engaging shank 40 relative to the receiver member 60. The outer surface 77 of the walls 74 may have a circular shape. The outer surface 77 may be smooth or have threads to engage the sleeve member 20 described below.

In other exemplary embodiments, the expandable socket 72 may be tapered or may have any other shape that allows adjustment of the head 42 of the shank 40 relative to the receiver member 60. In the exemplary embodiment, the bone anchor assembly 10 is a polyaxial bone anchor assembly. The bone-engaging shank 40 when assembled within the receiver member 60 may be pivoted to one or more angles relative to the receiver member 60.

Referring to FIG. 1C, sleeve member 20 of the bone anchor assembly 10 is positionable about the expandable socket 72 of the receiver member 60 to inhibit expansion of the expandable socket 72. The sleeve member 20 extends from a proximal end 12 to a distal end 14. The sleeve member 20 is sized and shaped to retain the head 42 of the shank 40 within the receiver member 60 by inhibiting expansion of the expandable socket 72 after the head 42 of the shank is in place. The sleeve member 20 may have a generally spherical shape complementary to the shape of the receiver member 60. The sleeve member 20 may have an inner surface 26 contoured for engaging the outer surface of the walls 74 of the expandable socket 72. In one embodiment, the inner surface 26 may be threaded to engage threads on the outer surface of the expandable socket 72. Alternately the inner surface 26 may be smooth providing a press fit between the expandable socket 72 and the sleeve member 20.

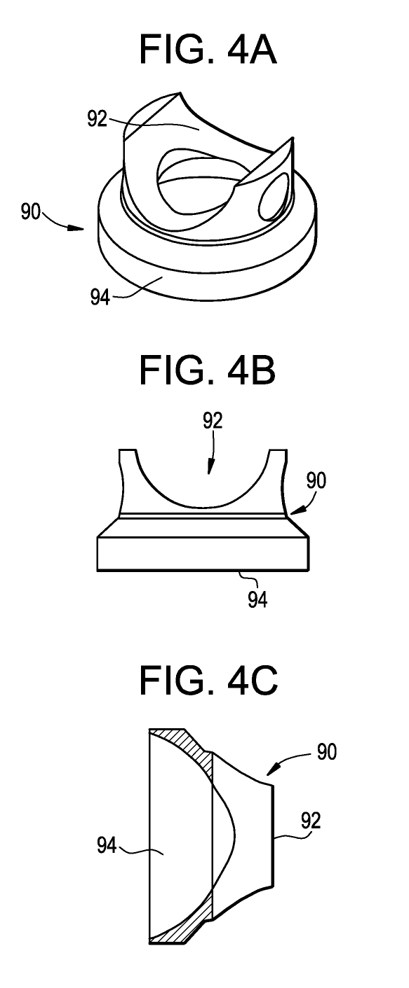

The bone anchor assembly 10 may optionally include a compression member 90 as shown in FIGS. 4A-C positionable within the receiver member 60 between the spinal connection element and the head 42 of the bone engaging shank 40. As illustrated in FIGURE IC, the compression member 90 may be positioned within the recess 68 between the spinal rod 80 and the head 42 of the shank 40. In the exemplary embodiment, the compression member 90 may have a proximal first surface 92 for engaging the spinal connection element and an opposing distal second surface 94 for engaging the head 42 of the shank.

The exemplary bone anchor assembly 10 may include a closure mechanism 100 that secures the spinal connection element to the bone anchor assembly. Referring to FIG. 1A, the closure mechanism 100 secures the exemplary spinal rod 80 within the recess 68 of the receiver member 60. The closure mechanism 100 may engage the first end 62 of the receiver member 60 or, in other exemplary embodiments, may engage other portion(s) of the receiver member 60. The exemplary closure mechanism 100 is an internal setscrew that engages an inner surface of the first end 62 of the receiver member 60. For example, the closure mechanism 100 may have external threads 102 that engage internal threads 104 provided on the first end 62 of the receiving member 60. Distal advancement of the closure mechanism 100 into engagement of the spinal rod 80, seats the spinal rod 80 in the proximal surface 22 of the compression member 90. The compression member 90 then is advanced onto the head 42 of the bone-engaging shank 40 thereby fixing the relative movement of the head 42 in relation to the receiver member 60. In one embodiment, the major diameter of the bone-engaging shank 30 may be greater than the diameter of the closure mechanism 100.

One skilled in the art will appreciate that other types of closure mechanisms may be employed. For example, an external closure mechanism positionable around the outer surface of the legs 76A, 76B of the receiving member 60 may be employed. In other exemplary embodiments, the closure mechanism may comprise an external and an internal closure mechanism, a non-threaded twist-in cap, and/or any other conventional closure mechanism.

The components of the bone anchor assembly may be manufactured from any biocompatible material, including, for example, metals and metal alloys such as titanium and stainless steel, polymers, and/or ceramics. The components may be manufactured of the same or different materials. In one exemplary method of manufacturing, the bone-engaging shank 40, the retaining member 20 and the receiver member 60 are separately constructed and assembled prior to implantation. The head 42 of the shank 40 is inserted proximally through the bore 64 to expand the expandable socket 72 of the receiver member 60. The sleeve member 20 is positioned around the expandable socket 72 to inhibit expansion of the expandable socket 72 and to retain the head 42 of the bone-engaging shank 40 within the receiver member 60. In one exemplary method the sleeve member 20 may be positioned about the expandable socket 72 by threading. If the sleeve member 20 is threaded into position around the expandable socket 72, the threads may be deformed or staked to provide additional retention of the sleeve member 20 to the receiver member 60. Alternately, the sleeve member 20 may be welded, swaged, press fit or staked in position around the expandable socket 72 of the receiver member 60.

While the large diameter bone anchor assembly and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

What is claimed:

1. A bone anchor assembly for engagement to a connection element comprising:
a bone-engaging shank having a head at a proximal end,
a receiver member having an opening at the proximal end for receiving the connection element and an expandable socket, the expandable socket expandable from a first configuration in which the diameter of the expandable socket is less than the size of the head of the shank to a second configuration in which the diameter of the expandable socket is greater than or equal to the size of the head of the shank; and
a sleeve member positionable about the expandable socket, the sleeve member having an inner surface sized and shaped to inhibit expansion of the expandable socket from expanding when positioned about the expandable socket and thereby retains the head of the shank within the receiver member.

2. The bone anchor assembly of claim 1, wherein the head of the shank has a generally spherical shape.

3. The bone anchor assembly of claim 2, wherein the expandable socket has an inner surface generally spherical shaped to accommodate the head of the shank.

4. The bone anchor assembly of claim 1, wherein the expandable socket has a threaded outer surface.

5. The bone anchor assembly of claim 4, wherein the inner surface of the sleeve member is threaded.

6. The bone anchor assembly of claim 1, wherein the expandable socket has slits.

7. The bone anchor assembly of claim 1, wherein the sleeve member has a generally semi-spherical shape.

8. The bone anchor assembly of claim 1, further comprising a compression member.

9. The bone anchor assembly of claim 1, further comprising a closure mechanism wherein the diameter of the head of the shank is greater than the closure mechanism.

10. The bone anchor assembly of claim 9, wherein the major diameter of the shank is greater than the closure mechanism.

11. The bone anchor assembly of claim 1, wherein the major diameter of the shank is greater than the diameter of the opening of the receiver member.

12. A method of assembly of a bone anchor comprising:
expanding an expandable socket of a receiver member to receive a head of a bone-engaging shank; and
positioning a sleeve member around the expandable socket of the receiver member to inhibit expansion of the expandable socket and to retain the head of the bone-engaging shank within the receiver member.

13. The method of claim 12, wherein the sleeve member is positioned by threading around the expandable socket of the receiver member.

14. The method of claim 12, wherein the sleeve member is press-fit around the expandable socket of the receiver member.

15. The method of claim 14, further comprising:
swaging, welding or staking of the sleeve member to the expandable socket of the receiver member.
